(19) United States
(12) Patent Application Publication
(10) Pub. No.: US 2026/0053622 A1
(43) Pub. Date: Feb. 26, 2026

(54) Annuloplasty Band

(71) Applicants: Henry Tannous, Hanover, NH (US); Nour Tannous, Hanover, NH (US); Joy Tannous, Hanover, NH (US)

(72) Inventors: Henry Tannous, Hanover, NH (US); Nour Tannous, Hanover, NH (US); Joy Tannous, Hanover, NH (US)

(21) Appl. No.: 18/971,959

(22) Filed: Dec. 6, 2024

Related U.S. Application Data
(63) Continuation-in-part of application No. 18/809,426, filed on Aug. 20, 2024.

(51) Int. Cl. A61F 2/24

(52) U.S. Cl. CPC A61F 2/2445; A61F 2/2418; A61F 2210/009; A61F 2220/0008; A61F 2220/0075; A61F 2230/0006; A61F 2250/0003

(57) ABSTRACT

An annuloplasty band is provided. The band is sized and configured to be attached to an annulus of a heart valve. Later, if needed, a cable may be attached to opposing ends of the annuloplasty band at connectors on each end of the band. This forms a more secure anchor and a closed shape of the band. A percutaneous TMVR or TTMR heart valve replacement may then be attached to the closed band using it as a framework for anchoring the valve.

### BACKGROUND OF THE INVENTION

The present disclosure relates generally to annuloplasty technology.

The heart is a hollow muscular organ with four pumping chambers: the left and right atria and the left and right ventricles. One-way valves between each of the chambers control the flow of blood in and out of the heart. The valves that control the blood flow between the atria and the ventricle are termed as Atrio-Ventricular Valves while the valves between the Ventricles and the outflow tracts are Outflow Tract/Semi-lunar Valves. The left atrio-ventricular valve is called the Mitral Valve, while the left ventricular outflow tract valve is called the Aortic Valve. Similarly, the right atrio-ventricular valve is called the Tricuspid Valve, while the right ventricular outflow tract valve is called the Pulmonary Valve.

Heart failure related to heart valve dysfunction is a common condition that may be due to the heart valves failing to function properly. Surgery to repair damaged valves is the method of choice over valve replacement in the current surgical era. Surgical repair techniques involve reconstruction or controlled alteration of the geometry of the native valve using implantable devices. One of the most common repair techniques used today by the surgeons to repair atrio-ventricular valve regurgitation is annuloplasty, in which the valve annulus is geometrically stabilized or reduced in size by suturing a prosthetic annuloplasty device onto the annulus. The annuloplasty devices are designed to roughly conform to the shape of the annulus, allow more ample leaflet coaptation to prevent leakage, while maintaining a good forward flow. These annuloplasty devices are generally made in different shapes, sizes and mechanical properties. A D-shaped annuloplasty device is the most common among the shapes with two important sub-categories being the full ring and the partial/incomplete ring (also called a band). The rings can be made rigid, semi-flexible and flexible depending on specific needs of the patient. A “partial ring”, an “incomplete ring”, and a “band” will be used interchangeably in this document.

In the current era, implantation of these rings requires surgical interventions with open-chest surgery. Placement of a full ring around the mitral valve provides more support to the valve annulus. The full ring is advantageous because it can act as an anchor to install a future transcutaneous valve replacement without reopening the patient's chest, in case the need arises as the patient gets older or the repair fails. However, full rings have been decreasing in popularity and usage nationally over the past 2 decades. An alternative to a full annuloplasty ring is a “band” or “partial ring”. A band is faster to implant, carries a lower risk of injuring surrounding structures, preserves the aortomitral curtain flexibility, and is easier to insert through small incisions like robotic mitral surgery. Moreover, 100% of tricuspid prosthesis are bands (not full rings) to avoid injuring the conduction system of the heart. The downside to the bands is that, because they are not a fully closed shape, they cannot be used as anchors for a future percutaneous valve replacement (TMVR or TTVR) if the need arises. All these patients will need another open surgery to replace their valves. It is estimated that between 2,000-4,000 patients in the US get mitral valve bands per year. An additional 5,000-10,000 patients get tricuspid bands yearly.

As mentioned above, the increased trend of using a band over a complete ring has been attributed to an easier implant, preservation of the aorto-mitral curtain flexibility, and the advantage of introducing a smaller device through minimally invasive incisions during mitral repairs.

TMVR (Transcutaneous Mitral Valve Replacement) is a less invasive way to replace the mitral valve without opening someone's chest. For patients with a history of a previous mitral valve repair and a recurrent mitral disease, only those with a complete annuloplasty ring are candidates for a TMVR (due to enough circumferential support). Patients with bands are denied this less invasive replacement option, and a second open heart surgery is needed.

Therefore, what is needed is an annuloplasty band which may be made suitable for a framework/anchor for a percutaneous valve replacement.

### SUMMARY OF THE INVENTION

The subject matter of this application may involve, in some cases, interrelated products, alternative solutions to a particular problem, and/or a plurality of different uses of a single system or article.

In one aspect, an annuloplasty band is provided. The band comprises a body having a first end and a second end, and defining a gap between the first end and the second end. In some embodiments, the body comprises a core, and a sheath covering at least a portion of the core. The sheath may be able to receive sutures for connection to an annulus of a heart valve. The band further has a first connector at the first end, and a second connector at the second end. These connectors are formed to be capable of being connected by a cable spanning the gap between the first end and the second end, thereby forming a complete ring from the band when the cable is attached to the two connectors.

In another aspect, an annuloplasty band is provided. The band comprises a core having a first end and a second end, and defining a gap between the first end and the second end and a sheath covering at least a portion of the core, the sheath able to receive sutures for connection to an annulus of a heart valve. The first and second ends of the band (or core) are connected by a cable spanning the gap between the first end and the second end, thereby creating the full ring comprising the band and connected cable.

In yet another aspect, a method of installing a transcutaneous mitral valve replacement valve or transcutaneous tricuspid valve replacement valve in a heart is provided. The method involves joining a first end of a previously installed annuloplasty band with a second end of the annuloplasty band by a cable extending across a gap between the first end and second end without removing the band from the body of the patient. Further, the method involves attaching a replacement heart valve to a sheath of the annuloplasty band. In some aspects, the step of joining the first end of the annuloplasty band and the second end of the annuloplasty band comprises the steps of passing a tool and the cable through a vessel such as an artery or vein of a patient, and connecting the cable using the tool.

### BRIEF DESCRIPTION OF DRAWINGS

FIG. 1 provides an elevation view of a prior art annuloplasty band.

FIG. 2 provides a view of a first step of one embodiment of the present disclosure.

FIG. 3 provides a view of a second step of one embodiment of the present disclosure.

FIG. 4 provides a view of a third step of one embodiment of the present disclosure.

FIG. 5 provides a view of a fourth step of one embodiment of the present disclosure.

FIG. 6 provides a view of a fifth step of one embodiment of the present disclosure.

FIG. 7 provides a view of a first step of a second embodiment of the present disclosure.

FIG. 8 provides a view of a second step of the second embodiment of the present disclosure.

FIG. 9 provides a view of a third step of the second embodiment of the present disclosure.

FIG. 10 provides a view of a fourth step of the second embodiment of the present disclosure.

FIG. 11 provides a view of a fifth step of the second embodiment of the present disclosure.

FIG. 12 provides an elevation view of an embodiment of the present disclosure.

FIG. 13 provides an elevation view of another embodiment of the present disclosure.

FIG. 14 provides an elevation view of yet another embodiment of the present disclosure.

FIG. 15A provides a view of another embodiment of the present disclosure.

FIG. 15B provides a view of an embodiment of a step of yet another embodiment of the present disclosure.

FIG. 15C provides a view of an embodiment of another step of yet another embodiment of the present disclosure.

FIG. 15D provides a view of an embodiment of another step of yet another embodiment of the present disclosure.

FIG. 16 provides a view of a connection of the present disclosure.

FIG. 17 provides a perspective view of still yet another embodiment of the present disclosure.

FIG. 18 provides a side view of the embodiment of FIG. 17.

FIG. 19 provides a view of an inflation step of one embodiment of the present disclosure.

FIG. 20 provides a view of a finished installation of an embodiment of the present disclosure.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

The subject of the present disclosure converts an annuloplasty band into a functionally full mitral/tricuspid ring to allow a valve-in-ring therapy. None of the valve prosthesis currently available serve that goal. The present disclosure allows patients with a partial ring to be offered the less invasive Valve-In-Ring option. In the mitral space it is referred to as TMVR (Transcutaneous Mitral Valve Replacement) and in the tricuspid space it is referred to as TTVR (Transcutaneous tricuspid valve Replacement). Once the completed ring is formed, it is structurally very different from prior art rings since approximately one third of it is made of a new cable. In one aspect, existing prior art bands may be converted into full rings as disclosed herein. In another aspect, new inventive bands are disclosed which are specifically made to support a connection of the cable at each end.

Generally, the present disclosure relates to an annuloplasty band (also referred to in the art as a “partial ring”) configured for implantation in the annulus of a heart valve, namely the mitral valve or tricuspid valve. Incomplete or partial rings will be referred to as “bands” throughout this document. This band is structured such that it may later have a cable connecting the ends of the band, making it suitable for use as a framework for a percutaneous heart valve replacement. Typically, the band has a connector structure at each end to receive the cable or otherwise attach to the cable directly or indirectly. Such bands are typically constructed from a core material, such as silicone or metal, that determines its rigidity (flexible, semirigid, or rigid). This core material may be sheathed in a cloth material, for example Dacron or polyester, through which sutures are placed to connect the band to the body-namely the annulus of a heart valve.

Notably the cable contemplated herein is a different structure and separate from the band material. The cable is formed as an elongate and flexible structure able to join ends of the band. The cable may be made of, for example, metal, cloth, plastic, and the like. This cable is a distinct and separate piece from the band, and is installed after installation of the band, in many cases years later.

While certain examples of the connector are disclosed herein, it should be understood that the connector may be any structure that allows for connection with the cable directly or via intermediate parts, without straying from the scope of this disclosure.

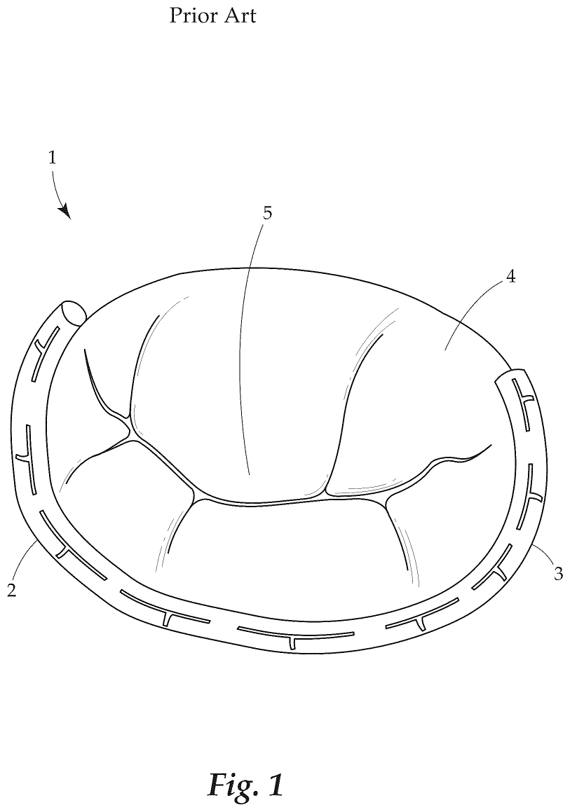

Turning now to FIG. 1, a prior art view of an embodiment of an annuloplasty band is provided. The band 1 has a body 2 comprising a core material and a sheath through which sutures 3 may pass to join the band 1 to the patient's body. The band 1 is attached to the annulus 4 of the heart valve 5, forming the annuloplasty. As noted above, using a band over a complete ring has been attributed to an easier implant, preservation of the aorto-mitral curtain flexibility, and the advantage of introducing a smaller device through minimally invasive incisions during mitral repairs. While a mitral valve is shown in the following figures, it is to be understood that the same procedures and structures may be applied to a tricuspid valve as well, without straying from the scope of the present disclosure.

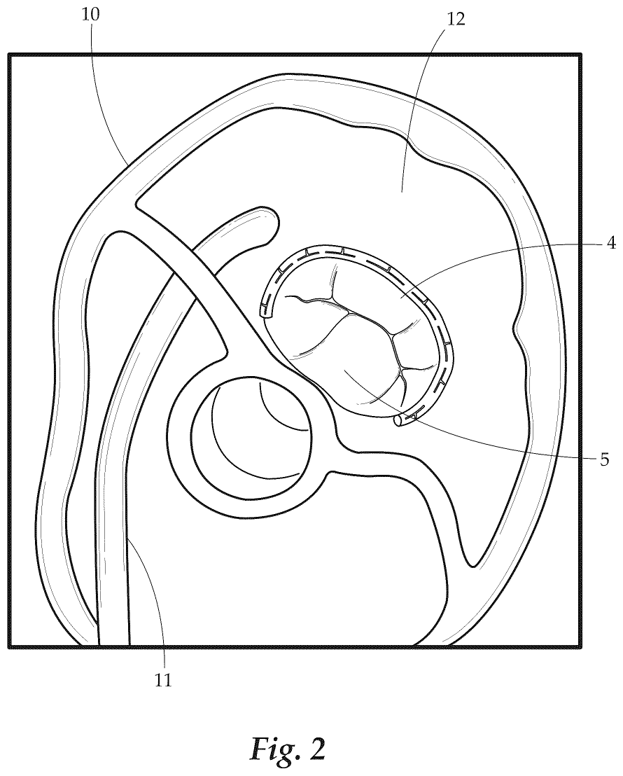

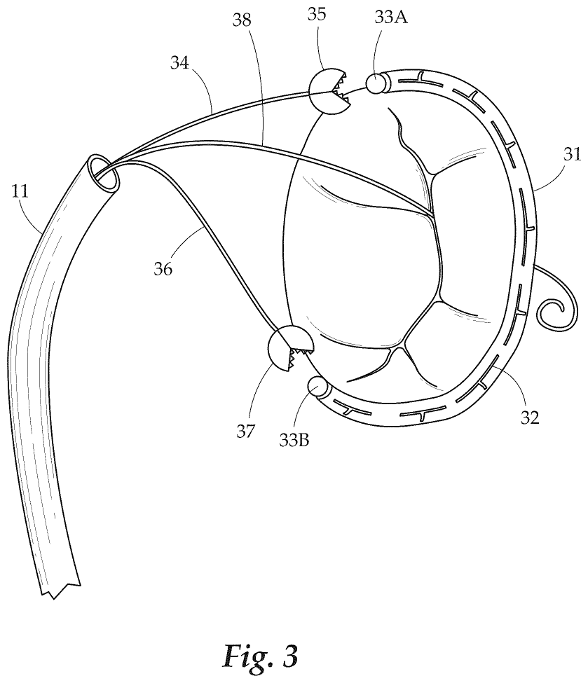

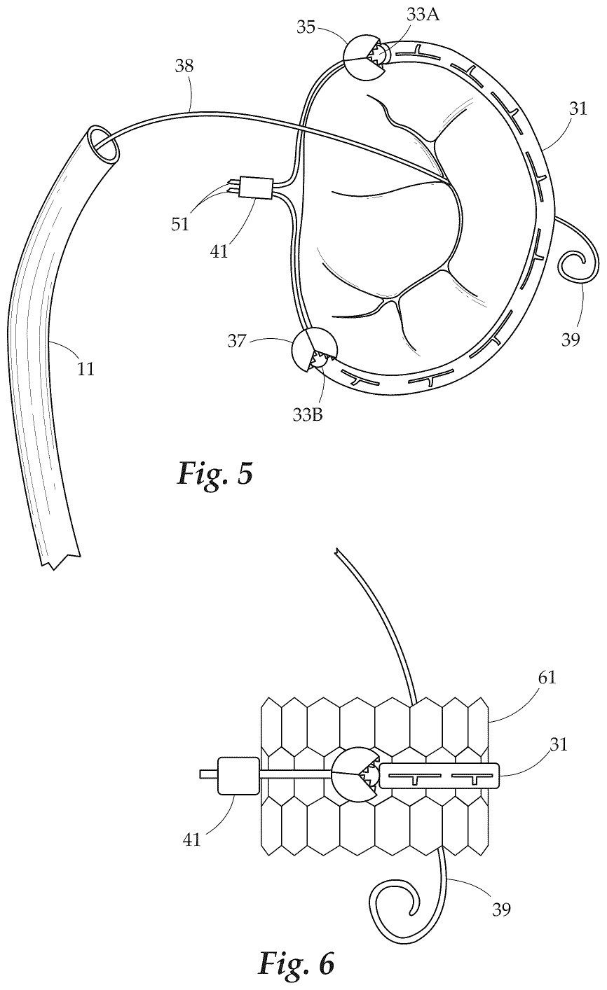

Turning to FIGS. 2-6, one embodiment of conversion of an annuloplasty band to a closed shape annuloplasty ring (a “complete ring”) is provided. This embodiment shows a two-cable technique. Two cables may be inserted into the body through the venous system via a delivery catheter 11 and passed into the right atrium 12 (or the left atrium through the Interatrial Septum of the heart) of heart 10 near the valve 5. As seen in FIG. 3, The cables 34, 36 may be made of metal, cloth, plastic, or the like. In one embodiment, biting clips (with jaws) shown as elements 35 and 35 or piercing needles (not shown) allow the securing of the cable 34, 36 to both ends of the band 33A, 33B. Of course, other connection structures such as caps, clamps, snaps, ties, and the like may be used to join the cable to band end without straying from the scope of the invention. In a particular embodiment, connection of the cables to the band ends may be done under fluoroscopy and echo guidance. The band 31, in many embodiments, comprises core and a sheath 32 around at least part of the band 31. Sheath is sutured to the annulus 4 of the heart valve 5. A guidewire 38, 39 is shown that will guide the TMVR/TTVR valve into place after the ring is completed. In some embodiments, as connected, the cable and band form a “D” shaped ring, but of course other shapes and configurations are within the scope of this disclosure.

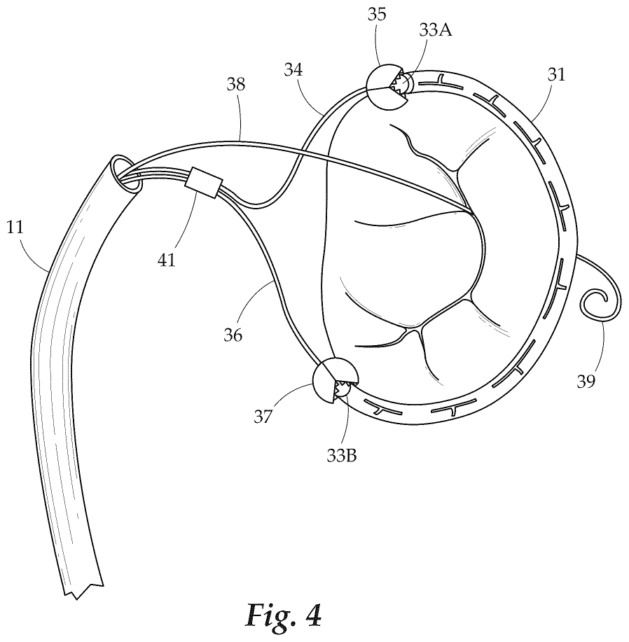

As seen in FIG. 4, once both cables are secured and locked to the band, a joining clip 41 such as a crimpable joining clip may be placed on both cables 34, 36 outside the body, guided under, e.g. fluoroscopy and echo guidance to the right length, and crimped to connect both cables securely together, thereby bridging the two ends 33A, 33B of the band 31 and complete the ring. Both cables 34, 36 are then cut proximal to the clip 41. The completion of the ring from the band then allows for a TMVR/TTVR/Valve-In-Ring 61 to be supported by a complete ring. This device 61 may then be installed using known methods in the art, as seen in FIG. 6.

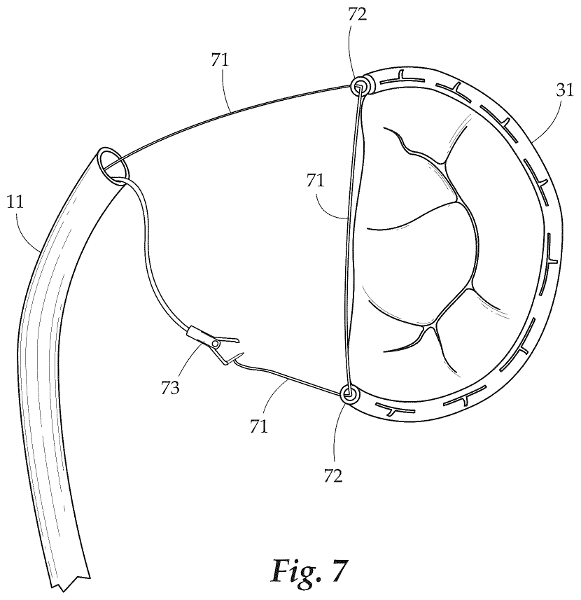

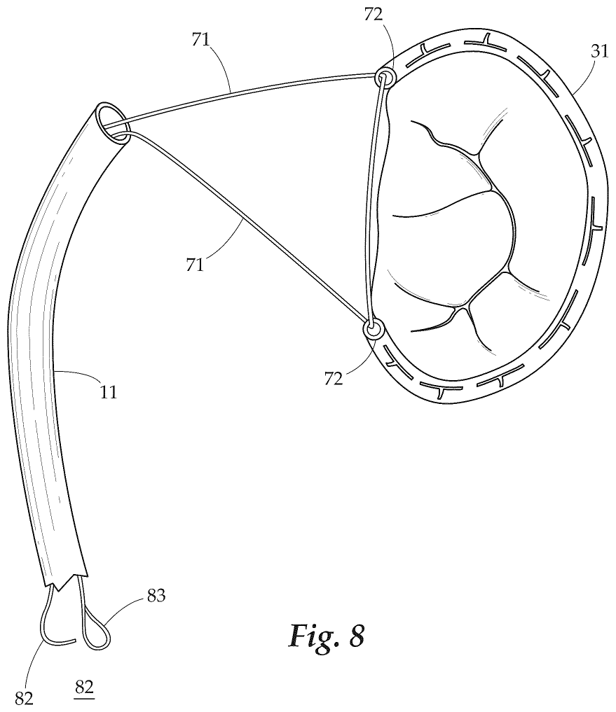

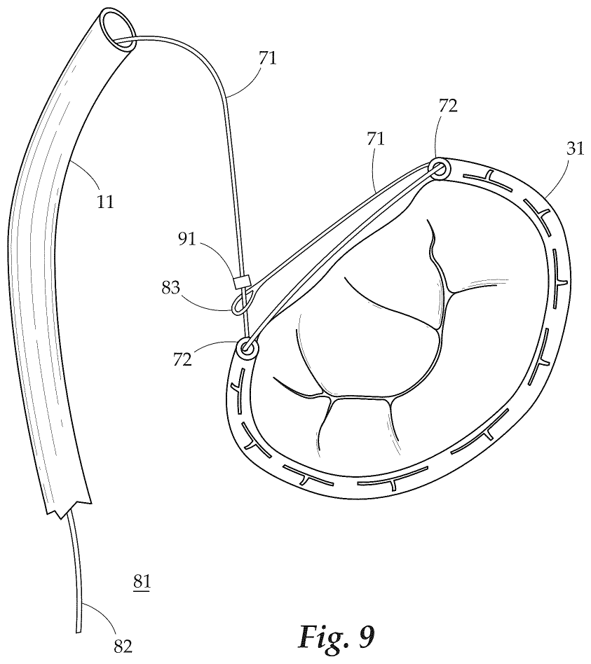

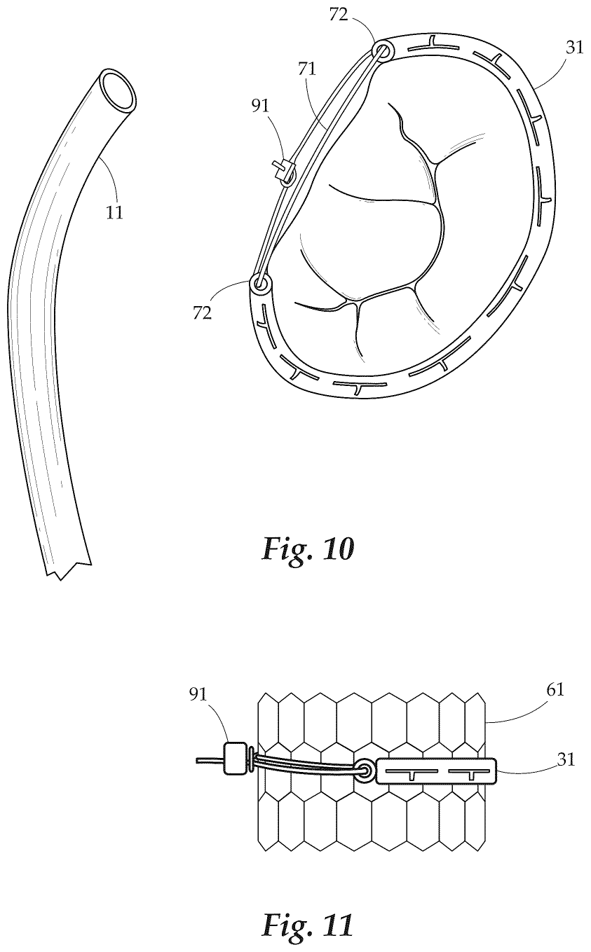

Turning to FIGS. 7-11, another embodiment of conversion of an annuloplasty band to a closed shape annuloplasty ring (a “complete ring”) is provided. This technique uses a single cable to join the ends of the annuloplasty ring to form a closed shape complete ring to support a replacement heart valve. As seen in FIG. 7, a cable 71 attached to a needle 73 is introduced in a similar fashion into the atrium via delivery catheter 11 and threaded through both ends of the band 31. In this view, band 31 comprises two loops 72, one at each end. In other embodiments, the needle 73 may thread cable 71 through a sheath or other pierceable portion of the band 31. As seen in FIG. 8, once the cable 71 is through both end loops 72, it is externalized from the body 81 and, while outside the body a small loop 83 at the end of the cable is threaded by the other end 82 of the cable 71 (end passed through the loop). As seen in FIG. 9, the loop 83 is pushed into the patient and guided to the left atrium creating a double cable connection between the two ends 72 of the band 31. A clip 91 is slid along the cable 71 and then secured in place on the cable once the right length is achieved. The clip 91 is sized such that it is larger than the opening of the loop 83 and cannot pass through. Once the clip 91 is secured, excess cable 71 is clipped, leaving the completed full ring as seen in FIG. 10. The completion of the ring from the band then allows for a TMVR/TTVR/Valve-in-ring 61 to be supported by a complete ring. This device 61 may then be installed using known methods in the art, as seen in FIG. 11.

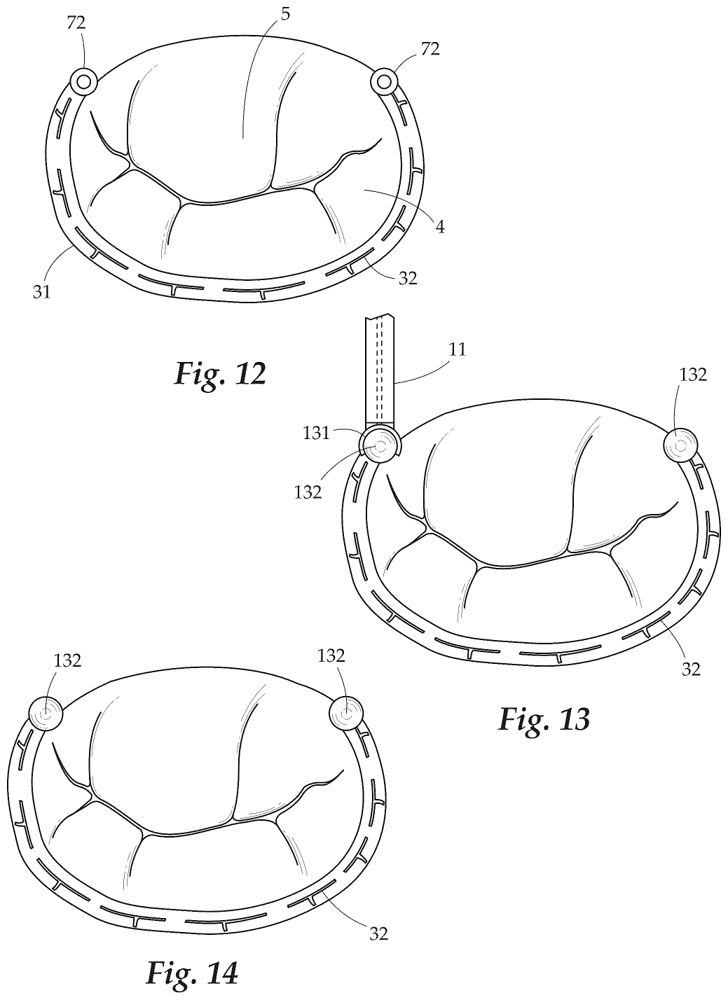

FIG. 12 provides a view of an embodiment of an annuloplasty band contemplated herein. In this view, the band is shown installed to an annulus of a heart valve. A band 31 has a body portion 32 which allows sutures to pass through and connect to the annulus 4 of a heart valve 5. In typical embodiments, the body 32 comprises a core to provide shape and form to support the valve annulus 4, as well as a sheath of fabric or other pierceable material to receive sutures for connection to the body. The body 32 may be formed in the curved position shown, in many embodiments, or may bend to adopt the shape of the installation, and in certain embodiments may apply a force against the annulus 4 when installed. In addition, each end has a connector for connecting with the cable. In this embodiment, the connector comprises a loop 72 through which a cable may pass allowing the band 31 to be later converted to a full ring in the event that the patient requires a heart valve replacement in the future. Of course, other connector structures may be used to engage with the cable without straying from the scope of this disclosure. As such, the band 31 can be installed for annuloplasty purposes and, if needed, can later be converted to a full ring to support a percutaneous heart valve replacement without removal of the band, and in a minimally invasive procedure avoiding any chest incisions. This greatly reduces risk to the patient, reduces recovery time and patient suffering, and provides a more reliable and secure procedure for heart surgery.

FIGS. 13 and 14 provide views of an embodiment of the band contemplated herein. Each end of the band 31 has a connector for connecting with the cable. Here the connector comprises two beads 132 at each end. The beads 132 are typically rounded or spherical shape, but may be any shape and configuration without straying from the scope of this invention. Typically, the bead 132 will be formed to have a wider portion and a reduced width portion closer to the band 31 center. This allows a cap 131 connected to an end of the cable 133 to extend over the bead 132 and be crimped or otherwise secured onto the bead 132 thereby connecting cable 133 to an end of the band 31. In some embodiments, the bead 132 may be covered by a cover (not shown) when first installed. This cover may be removed prior to connection of the cap 131 to the bead 132. In certain embodiments, the bead 132 may be formed of or may comprise a magnet or magnetic material. In such an embodiment, the cap 131 may also comprise a magnet or magnetic material. In particular the magnet or magnetic material may be at the rear of cap 131 to pull the whole cap 131 over the bead 132. As such, during connection, cap 131 may be drawn via a magnetic attraction to bead and preferably onto and over bead 132 into a proper position. This greatly eases the connection process. Of course, other connector structures, including other magnetically enhanced connectors, may be used to engage with the cable without straying from the scope of this disclosure.

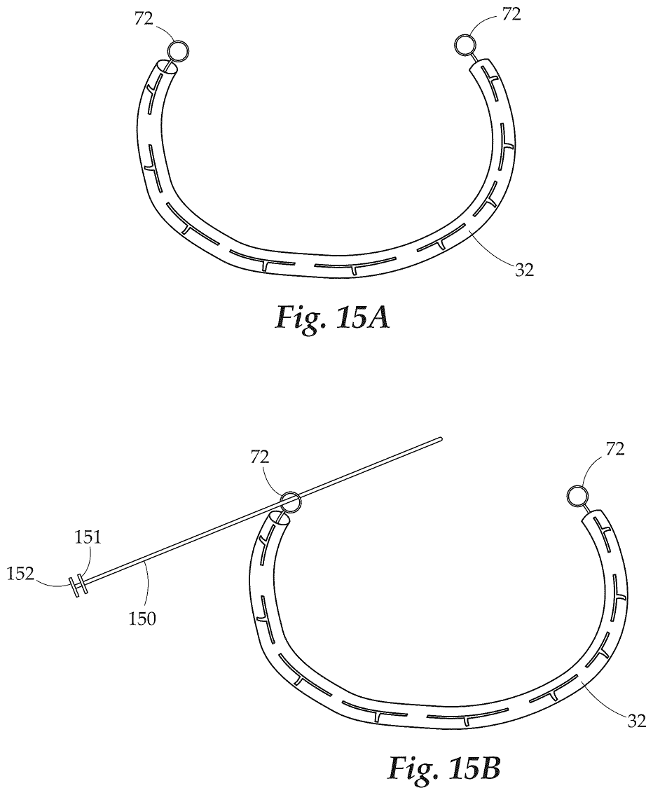

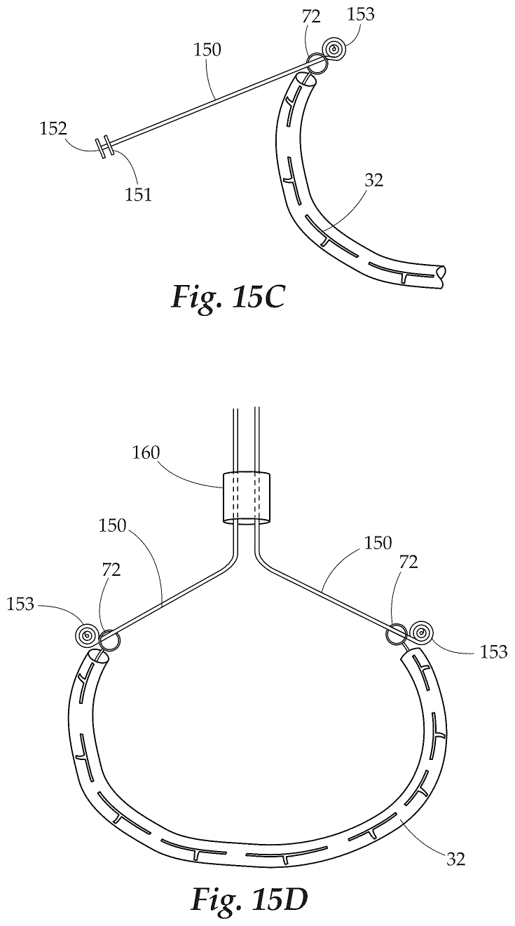

FIG. 15 A-D provide a view of another embodiment of the band contemplated herein. The band is formed of a body 32, and includes elements as described relating to other embodiments, above. The band has loops 72 at each end. In this embodiment, a locking cable/wire which has a bendable or deformable tip is used to connect the end loops 72. The loops 72 are sized to allow the cable to pass through, allowing connection of the ends of the band such that the band can be later converted to a full ring in the event that the patient requires a heart valve replacement. In this embodiment, a cable 150 with deformable end 153 is shown. This cable 150 can be manipulated by a tool to cause its end 153 to deform/change shape. In the embodiment shown, the cable has a finger hold 151 and actuator 152 which, when depressed, causes the deformable end 153 to actuate, forming it into an anchor.

The deforming or other shape change of the end 153 of cable 150 may be by way of i.e. a bend or deform, such as by coiling, enlarging, changing shape, angling, bending, or the like-as seen in FIG. 15C. Of course, other structures and systems able to deform the end of the cable 150 may be used and are contemplated herein. The deformable end 153 could be enlarged by being filled with fluid (either permanently or temporarily) could have tension applied to it via e.g. an internal wire/cord; could have tension released from it, and the like. Finger hold 151 and actuator 152 are positioned outside of the body and, as discussed throughout, the process may be performed in a minimally invasive fashion via heart access from an artery or small openings in the chest, or other minimally invasive heart surgery methods compared to an open heart surgery. Thus, an installed band such as that of FIG. 15A may be converted to a full ring by having the cable 150 passed through one ring 72, and then the cable 150 is deformed creating an anchor 153 that cannot fit through ring 72 by depressing the actuator 152. Actuation to create the anchor 153 is permanent, such that the anchor 153 does not disappear after the procedure is finished. The process is repeated with a second cable 150 in the other ring 72. Then the two cables 150 are held together via, e.g. a joining clip 160 or other manner. The joining clip 160 is slid from outside the body until it holds the two cables 150 at a proper tension (which are connected to rings via anchor 153), and the excess wire behind the clip 160 is cut, thus forming a full ring from the partial band.

In another embodiment similar to that of FIGS. 15A-D, in embodiments of the band having loops at each end, a cable having a free end and a pre-formed anchor is at an opposite end. The anchor is formed as a structure which is too large to fit through the opening of ring 72, such as a bead, cap, block, crimped tab, loop or other arrangement of the cable, and the like, thereby preventing passage of the cable completely through the ring. In an installation embodiment, the cable free end may be threaded through a ring and then externalized. Once drawn fully, the anchor will meet the loop and be prevented from further motion, as the anchor cannot pass through the loop. This process may be repeated for the second loop at the other end of the band with a second cable having an anchor at one end, or the cable free end may go through both rings. and then the cable can be crimped or otherwise secured as discussed above-with the anchor engaging with the ring holding the cable in place.

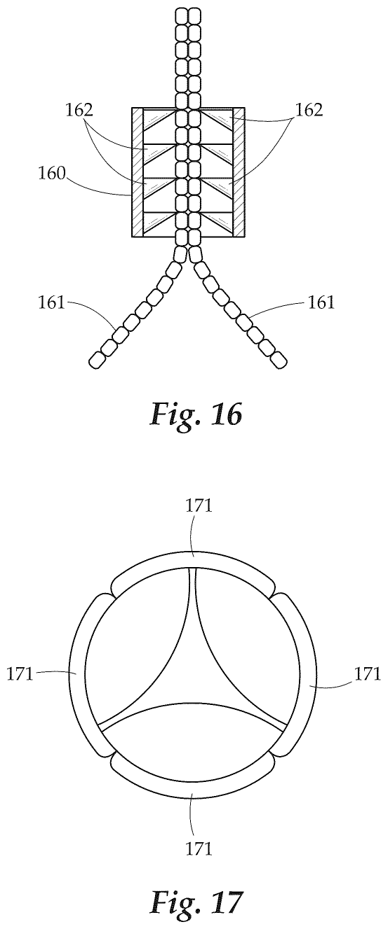

FIG. 16 provides a view of one embodiment of a locking clip 160. The locking clip 160 shown is an alternative to a crimpable clip discussed above or other connecting structures to hold two cables/wires together. The clip 160 shown is formed of a body having open top and bottom and which defines an internal opening that has locking teeth 162 extending into a center. The clip is able to receive free ends of cable 150 and hold them together and in place. The teeth 162 are shaped having a ramp facing in the same direction and flat top side. In some cases, the teeth are formed of a deformable material such as plastic or metal which is able to deform somewhat away from the ramp side. This allows fairly easy sliding of cables 161 in a first locking direction (towards the ramps of the teeth 162) by either slipping past the teeth or deforming the teeth, but prevents or limits movement in an opposite unlocking direction towards the flat side of the teeth 162. In some embodiments, the locking direction movement may be via sliding, or via a rotational motion. In some embodiments, the clip may be movable in the opposite unlocking direction via rotation, but can allow for a sliding of the wires in the locking direction. Rotational movement may be particularly advantageous in braided cable embodiments allowing the teeth to engage with the twisted braids of the cable. The locking clip 160 may be used in any of the embodiments disclosed herein, or other non-disclosed embodiments, as a tool to securely connect two wires or cables together.

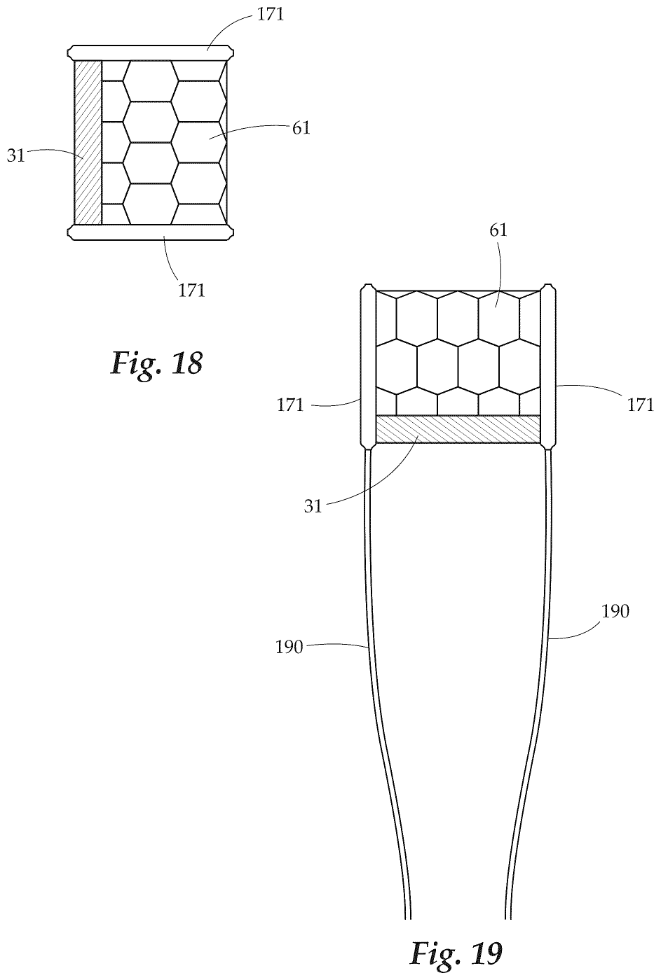

FIGS. 17-19 provide views of an embodiment of a TMVR/TTVR/Valve-in-ring of the present disclosure. The embodiment disclosed has structures similar to traditional TMVR/TTVR/Valve-in-ring, including a body or “frame” 61 and replacement valve component 5, often referred to a leaflets which open and close to regulate blood flow. Some embodiments include a sealing skirt, though these have limited effectiveness in preventing leaks around the replacement valve. In addition, the valve-in-ring frame 61 includes a plurality of inflatable or otherwise fillable pouches 171. After deployment of the valve, one or more of the plurality of pouches 171 may be filled to decrease leakage around it. Multiple pouches and/or one or more multi-compartmentalized pouches allow for filling only in a targeted area to treat a leak in a specific spot. The valve is placed in the body and is shown having frame 61 anchored by band 31 which has been converted to a full ring as discussed throughout this disclosure. The valve has the pouches 171 connected thereto, typically around the perimeter on the outside of the valve frame 61. The inflation of pouches 171 occurs via a port (see FIG. 18) allowing for detachable connection to a catheter 190. The catheter 190 may connect with the port to deliver gas or liquid and inflate one or more pouches 171 or compartments thereof. Typically liquid is used as it is safer in case of a leak and is more predictably controllable and deliverable in volume. A connector on the port provides a seal to prevent deflation by having, for example, a valve, check valve, seal, closure, or the like at or near the detachable connection port of the inflatable pouch 171. Inflation and sizing of the pouches 171 may be done at the time of valve placement, or after a period of time if/when a leak has been identified. Once installed, in the percutaneous space, the inflation may guided by echo and fluoroscopy (after the implantation) and it would target any leaking spots. In some embodiments of pouches 171 having a plurality of compartments, there may be a single port corresponding to each of the compartments, allowing for precise and controlled inflation at particular areas.

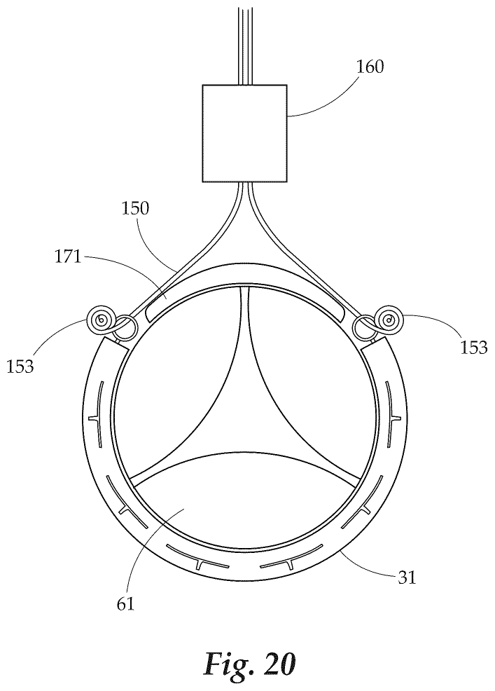

FIG. 20 provides a view of the valve embodiment shown in in FIGS. 17-19 attached to a band embodiment shown in FIG. 15 A-D. Band 31 has ends which are joined to form a full ring via cables 150 which have deformed anchors 153 ad the end, holding them to loops 72. The cables 150 are then joined to each other via clip 160, the locking clip shown in FIG. 16. The valve 61 comprises inflatable pouches 171 which can be filled with gas or liquid, typically liquid though not always. As can be seen, pouches 171 are in an inflated state, properly installed and fitted within the heart to prevent leaking around the valve.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is

1. An annuloplasty band comprising:
a core having a first end and a second end, and defining a gap between the first end and the second end;
a sheath covering at least a portion of the core, the sheath able to receive sutures for connection to an annulus of a heart valve; and
a first connector at the first end, and a second connector at the second end, the first and second connector capable of being connected by a cable spanning the gap between the first end and the second end;
wherein the first connector is a loop through which the cable may pass; and
wherein the first connector is a loop through which the cable may pass.

2. The annuloplasty band of claim 1 further comprising a removable cover over each of the first connector and second connector.

3. The annuloplasty band of claim 1 further comprising a magnet or magnetic metal on the first connector and second connector or on the annuloplasty band adjacent to the first connector and second connector.

4. The annuloplasty band of claim 1 wherein the band is sized converted to a full ring for installation for a percutaneous mitral valve or a tricuspid valve.

5. The annuloplasty band of claim 1 wherein the sheath is a fabric material.

6. An annuloplasty band assembly comprising the annuloplasty band of claim 1 and further comprising:
a cable spanning the gap between the first end and the second end connecting the first end and the second end, the cable comprising an anchor at a first end, the anchor being sized to prevent passage through the loop of the first connector and the loop of the second connector.

7. The annuloplasty band assembly of claim 6 wherein the anchor is formed at a deformable end of the cable.

8. The annuloplasty band assembly of claim 6 wherein the cable comprises a first cable portion comprising the anchor at a distal end engaged with the loop of the first connector, and a second cable portion comprising a second anchor at a distal end engaged with the loop of the second connector, proximal ends of the first cable portion and the second cable portion being joined together by a clip.

9. The annuloplasty band assembly of claim 8 wherein the clip is crimped.

10. The annuloplasty band assembly of claim 8 wherein the clip comprises a plurality of internal teeth engaging the first cable portion and the second cable portion.

11. The annuloplasty band assembly of claim 10 wherein each of the plurality of teeth has a sloped ramp facing a first direction and a flat face facing a second opposite direction.

12. The annuloplasty band assembly of claim 6 further comprising a Transcutaneous Mitral Valve Replacement valve or Transcutaneous tricuspid valve Replacement valve connected to the band.

13. A replacement heart valve comprising:
a frame;
leaflets within the frame; and
a plurality of inflatable pouches attached to the frame.

14. The replacement heart valve of claim 13 wherein the plurality of inflatable pouches are arranged around a perimeter of the frame.

15. The replacement heart valve of claim 13 wherein each of the plurality of inflatable pouches comprises a port.

16. The replacement heart valve of claim 13 wherein at least one of the plurality of inflatable pouches comprises a multi-compartment pouch.

17. The replacement heart valve of claim 16 wherein the multi-compartment pouch comprises one port in communication of each compartment, allowing inflation of each compartment individually.

18. The replacement heart valve of claim 15 wherein each of the ports of the plurality of inflatable pouches comprises a connector allowing removable connection of a catheter.

19. The replacement heart valve of claim 15 wherein each of the ports of the plurality of inflatable pouches comprises a check valve.

20. The replacement heart valve of claim 13 attached to an annuloplasty band, the annuloplasty band comprising:
a core having a first end and a second end, and defining a gap between the first end and the second end;
a sheath covering at least a portion of the core, the sheath able to receive sutures for connection to an annulus of a heart valve; and
a first connector at the first end, and a second connector at the second end, the first and second connector capable of being connected by a cable spanning the gap between the first end and the second end;
wherein the first connector is a loop through which the cable may pass;
wherein the first connector is a loop through which the cable may pass; and
a cable spanning the gap between the first end and the second end connecting the first end and the second end, the cable comprising an anchor at a first end, the anchor being sized to prevent passage through the loop of the first connector and the loop of the second connector.
